(19) United States
(12) Patent Application Publication
(10) Pub. No.: US 2026/0306092 A1
(43) Pub. Date: Oct. 8, 2026

(54) Surgical Constructs and Methods of Tissue Fixation

(71) Applicant: Arthrex, Inc., Naples, FL (US)

(72) Inventors: Loren D. CROOK, Fort Myers, FL (US); Lionel NEYTON, Naples, FL (US)

(73) Assignee: Arthrex, Inc., Naples, FL (US)

(21) Appl. No.: 19/571,806

(22) Filed: Mar. 19, 2026

Related U.S. Application Data
(60) Provisional application No. 63/783,279, filed on Apr. 4, 2025.

(51) Int. Cl. A61B 17/04

(52) U.S. Cl. CPC A61B 17/0401; A61B 2017/0464

(57) ABSTRACT

Knotless or knotted hybrid cerclage constructs and methods of tissue repairs are disclosed herein.

### CROSS-REFERENCE TO RELATED APPLICATIONS

The application claims priority to U.S. Provisional Patent Application No. 63/783,279 filed Apr. 4, 2025, the disclosure of which is hereby incorporated by reference in its entirety herein.

### BACKGROUND

The disclosure herein relates to the field of surgery and, more specifically, to hybrid suture and screw implants and associated methods of tissue repairs.

### SUMMARY

Surgical constructs, systems, assemblies, and methods of tissue repairs are disclosed. A hybrid construct can include a suture-based implant and a hard-body fixation device. A suture-based implant can include a fixation device in the form of soft suture anchors attached to a length of flexible coupler. A flexible coupler can be passed multiple times through the fixation devices to form a tensionable mechanism. A hard-body fixation device can be a non-suture-based device such a screw, pin, nail, hard-body anchor or fastener, or similar device.

Methods of tissue repairs are also disclosed. A first tissue may be approximated to a second tissue with a hybrid surgical construct that includes at least one tensionable construct and a hard-body fixation device such as a screw, pin, nail, hard-body anchor or fastener, or similar device. Free ends of a flexible coupler are passed multiple times through bodies of fixation devices (all-suture soft anchors). Ends of the flexible coupler exit one of the fixation devices and form adjustable, closed, tensionable, flexible loops that are interconnected. The tensionable construct is employed with a screw or similar device for in-line surgical repairs.

### BRIEF DESCRIPTION OF THE DRAWINGS

FIGS. 1 and 2 illustrate a surgical construct.

FIGS. 3 and 4 illustrate another surgical construct.

FIGS. 5 and 6 illustrate schematic views of the surgical construct of FIG. 3.

FIGS. 7-12 illustrate schematic steps of a repair using the constructs of FIGS. 1-6 (pull through method).

FIGS. 13-19 illustrate schematic steps of another repair using the constructs of FIGS. 1-6 (push through method).

FIGS. 20-22 illustrate schematic steps of another repair using the constructs of FIGS. 1-6 (pull through method with antegrade and retrograde leading sutures).

FIG. 23 illustrates a top view of a shoulder repair with the surgical constructs of FIGS. 1-6.

FIG. 24 illustrates a schematic top view of the shoulder repair of FIG. 23.

FIG. 25 illustrates a schematic lateral view of the shoulder repair of FIG. 24.

### DETAILED DESCRIPTION

The disclosure provides hybrid surgical adjustable loop constructs, suture loop mechanisms, in-line surgical repairs, and methods for securing a first tissue to a second tissue (for example, bone to bone) with a hybrid tensionable construct including adjustable, knotless, flexible, closed loops and a hard-body fixation device.

In an embodiment, the disclosure provides a hybrid suture/hard-body fixation device for tissue fixation. An orthopedic implant construct can attach or re-attach normal anatomical structures, tissue to tissue, bone to bone, and/or bone to soft tissue. The construct can include (i) a length of a single continuous flexible coupler in the form of suture, round, and/or flat suture attached to at least two fixation devices that act as soft buttons; and (ii) a hard-body fixation device such as a screw, pin, nail, hard-body anchor or fastener, or similar device.

A flexible coupler can be attached to a suture-based implant and a hard-body fixation device. Terminal ends of the flexible coupler exit one of the fixation devices of the suture-based implant to form a plurality of knotless, continuous, flexible, closed adjustable loops having an adjustable perimeter. The construct can be shrunk when both terminal ends are pulled. When the terminal ends are pulled, the construct shrinks, i.e., the perimeters of the knotless, continuous, flexible, closed adjustable loops decrease. The construct allows the user (for example, surgeon) to control the tension of the flexible coupler on a first tissue (for example, a first bone or first tissue fragment such as cartilage) to be attached to a second tissue (for example, second bone or second tissue fragment). The construct can be employed for surgical repairs with at least one hard-body fixation device such as a screw, pin, nail, hard-body anchor or fastener, or similar non-suture-based devices.

A tissue repair system may include (i) a suture-based implant comprising first and second fixation devices each in the form of a soft suture anchor; and a flexible coupler slidingly attached to the first and second fixation devices, wherein the flexible coupler includes two flexible ends (a first end and a second end) and wherein both first and second ends exit one of the first and second fixation devices at about same location; and (ii) at least one hard-body fixation device. The system can further include an inserter and a slotted cannulated guide. A flexible coupler can be of either round and/or flat design. The suture-based implant can consist essentially of suture such as ultrahigh molecular weight polyethylene suture. The at least one hard-body fixation device can be a screw, pin, nail, hard-body fastener, hard-body anchor, or similar device. A tissue repair system can provide knotted or knotless, in-line bone to bone fixation.

A hybrid construct can be employed to attach or re-attach normal anatomical structures, i.e., a first tissue to a second tissue, such as soft tissue, tendon, ligament, and/or bone, to each other and/or any combination of one another, by employing an in-line, knotless mechanism. A flexible coupler can be a fixed length of suture and/or tape. A hybrid construct can be employed as a stand-alone construct for fixation of first tissue to second tissue, for example, first bone to second bone. The hybrid construct has particular application to bone to bone fixation, such as bone graft fixation to glenoid for glenoid bone loss cases (Latarjet procedure, Bristow procedure, free bone block technique, etc.), eliminating the screw complications for one of the fixation points.

Cerclage constructs, systems, assemblies, kits and methods of cerclage repairs are also disclosed. A hybrid cerclage construct can create a repair with reduced metal cerclage and cabling (metal wires, cables, etc.) and/or reduced metal components (metal fittings, screws, buttons, suture-button constructs, etc.). A hybrid cerclage construct includes interconnected flexible strands that secure a first bone (bone graft or bone block) to a second bone (bone graft or bone block) with a simple and compact design. In an embodiment, a hybrid bone block cerclage repair system may include: (i) interconnected cerclage suture tapes, or combination of suture tapes and sutures, and all-suture soft anchors; and (ii) a hard-body fixation device such as a screw, pin, nail, hard-body anchor or fastener, or similar device. The hybrid bone block cerclage repair may secure a bone block or graft to the glenoid. The hybrid bone block cerclage construct is a flexible, tensionable construct that reduces the need for metal components in a fracture repair at one of the two fixation points. In certain implementations, the hybrid constructs may include one or more (i) all-suture constructs; and (ii) one or more hard-body fixation devices.

Bone cerclage methods are also disclosed. A first bone/graft is secured to a second bone/graft with a hybrid surgical construct that includes (i) at least one flexible strand attached to soft suture anchors; and (ii) at least one hard-body fixation device such as a screw, pin, nail, hard-body anchor or fastener, or similar device. In an embodiment, bone block cerclage is conducted with a hybrid flexible, tensionable surgical construct that includes a cerclage suture on one fixation point and a hard-body fixation device on another fixation point. In an embodiment, at least one flexible suture or tape (or combinations thereof) is passed through bones and secured to the bones by soft suture anchors. In an embodiment, at least one flexible coupler (flexible sutures or tapes or combinations thereof) is passed through bone tunnels to secure a first bone or first graft to a second bone or second graft, or to hold bony fragments together, to allow them to heal. In an embodiment, bone loss in glenohumeral dislocation or chronic glenohumeral dislocation/subluxation is reduced by securing a preshaped bone graft with a plurality of interconnected cerclage suture tape loops and soft suture anchors, in addition to a hard-body fixation device or similar non-suture-based device. A bone cerclage method may be a FiberTak® TightRope® hybrid fixation.

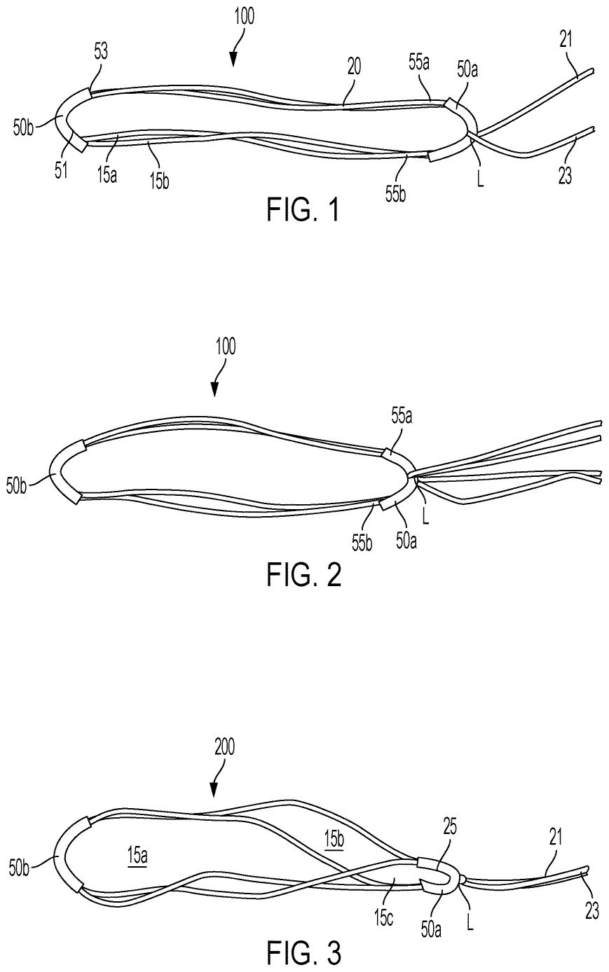

Referring now to the drawings, where like elements are designated by like reference numerals, FIGS. 1-6 illustrate structural elements of surgical assembly 100, 200 (cerclage construct 100, 200; construct 100, 200; all-suture suspensory fixation implant 100, 200; Fiber TightRope® all-suture suspensory fixation implant 100, 200; soft anchor bone fixation system 100, 200) formed of fixation devices 50a, 50b, 50c connected by flexible coupler 20.

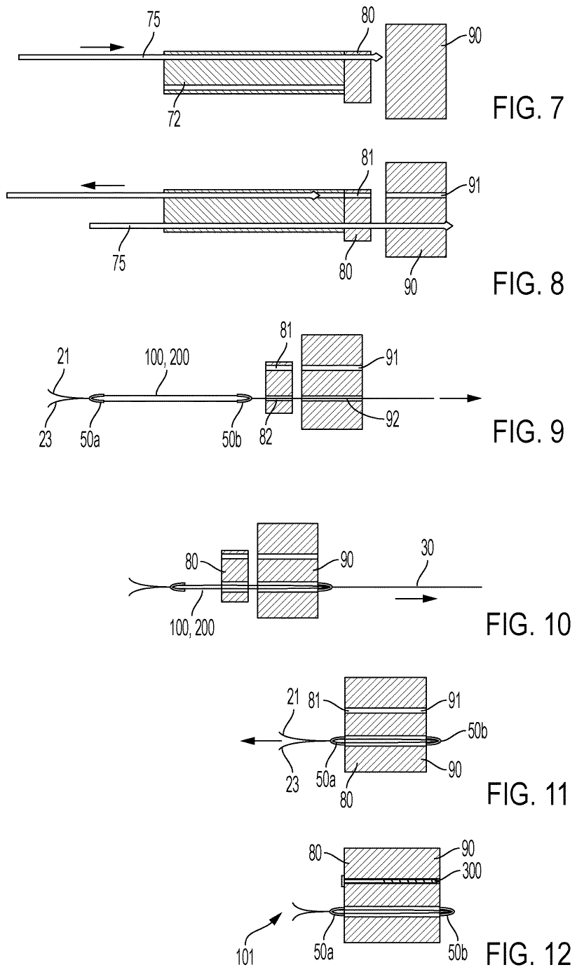

FIGS. 7-25 illustrate exemplary steps of tissue repair 101, 102, 103 with surgical assembly 100, 200 and exemplary hard-body fixation device 300.

As shown in FIGS. 1 and 2, assembly 100 includes two fixation devices 50a, 50b in the form of soft-suture anchors 50a, 50b. As detailed below, soft-suture anchors 50a, 50b are knotlessly attached to flexible coupler 20 by multiple loops. Each fixation device 50a, 50b can be in the form of a soft anchor (all-suture anchor, soft suture sheath, sheath, soft suture anchor, all-suture soft knotless anchor, implant, soft button) provided with a soft anchor sleeve 51 (sheath or tubular member 51) with two open ends 52, 53. At least one flexible coupler 20 extends through each of the soft anchor sleeve 51 (sheath 51) of each fixation device 50a, 50b.

Flexible coupler 20 can extend through the anchor sleeves in similar or different directions and/or orientations and/or locations. Flexible coupler 20 can extend through the whole length of each sleeve, i.e., enters one of the two open ends 52, 53 and exits other of the two open ends 52, 53. Details of an exemplary soft suture anchor with a soft anchor sleeve (sheath or tubular member) and flexible shuttling strands are set forth, for example, in U.S. Pat. No. 10,849,734 issued Dec. 1, 2020, entitled “Methods of Tissue Repairs,” the disclosure of which is incorporated by reference in its entirety herein.

In an embodiment, fixation devices 50a, 50b can be formed essentially of suture. In an embodiment, fixation devices 50a, 50b can be formed of braided suture that can include fibers of ultrahigh molecular weight polyethylene (UHMWPE). In an embodiment, fixation devices 50a, 50b can be formed of elastic suture. In an embodiment, fixation devices 50a, 50b can be formed of UHMWPE braided with polyester. UHMWPE is easy to splice and pierce; allows the construct to deform and remain in deformed shape; even at high PPI, it has minimal fraying and snagging; and has a low coefficient of friction. Polyester is difficult to splice without fraying suture; allows the construct to retain initial form; is difficult to pierce and pass TightRope® suture without fraying and snagging; and has a high coefficient of friction.

Flexible coupler 20 (tensionable construct 20; coupler 20; flexible material 20; flexible strand 20; flexible tape 20; suture 20) can be formed of one single continuous coupler in form of suture, either round and/or flat suture, for example, a suture strand or suture tape. Flexible coupler 20 is provided with two terminal ends, a first end 21 and a second end 23. Flexible coupler 20 can be a fixed length of suture and/or tape, such as suture tape.

In an embodiment, surgical assembly 100 can be formed by passing one of two terminal ends 21, 23 (for example, first end 21) of the flexible coupler through one of the soft-anchors 50a, 50b (for example, soft-anchor 50b, or distal anchor 50b). Terminal end 21 of flexible coupler 20 is passed through at least a portion of body 11 of fixation device 50b so that the two terminal ends 21, 23 of flexible coupler 20 reside outside the body 11 of fixation device 50b. Terminal end 21 is then passed through the other of the soft-anchors 50a, 50b (for example, soft-anchor 50a, or proximal anchor 50a). Terminal end 21 is passed a second time through body 11 of fixation device 50b and then enters the body of the fixation device 50a, at one of the two open ends 52, 53, through at least a portion of body 11 of fixation device 50a, and exits the fixation device 50a at about a mid-length of it, for example, at location L. Second terminal end 23 can then enter the body of the fixation device 50a, at the other of the two open ends 52, 53, through at least another portion of body 11 of fixation device 50a, and exits the fixation device 50a at about same location L.

The terminal ends 21, 23 are passed through proximal soft anchor 50a in different directions (for example, opposite directions) and exit the body of the soft anchor 50a at about same location L relative to the proximal soft anchor 50a. First and second terminal ends 21, 23 form first and second suture loops 15a, 15b. First and second terminal ends 21, 23 can also form first and second splices 55a, 55b. The passes and/or splices can be formed with suture passers and/or shuttle/pull devices and/or suture passing instruments such as needles, FiberLink™ loops, nitinol loops, or any suture passing device that includes an eyelet/loop for passing the flexible coupler 20. In an embodiment, the ends 21, 23 can terminate in a single tail, for example, the ends 21, 23 can be braided together, glued, spliced, connected and/or swedged together to form a single tail for ease of manipulation during the surgical procedures. The single tail can subsequently be cut to release the two free ends for additional steps.

Tensionable construct 100 can be shrunk when both terminal ends 21, 23 are pulled to decrease the perimeter of at least one of the flexible, closed, knotless, adjustable loops 15a, 15b.

One or more additional flexible strands 30 can be attached to construct 100 by securing the flexible strands to the soft anchors 50a, 50b or passing the flexible strands through any of loops 15a, 15b. Additional strands can be FiberWire® suture, TigerWire® suture, FiberTape® suture tape, among many others. FIG. 2 illustrates construct 100 with one additional flexible strand 30 attached to it. Although FIG. 2 illustrates flexible strand 30 attached to soft anchor 50a, it must be understood that the disclosure is not limited to this exemplary-only embodiment and, thus, the disclosure also contemplates embodiments where at least one flexible strand 30 is attached to far soft anchor 50b. In additional embodiments, one or more flexible strands 30 can be attached to each of soft anchors 50a, 50b. In other embodiments, a plurality of flexible strands 30 can be attached to one of the two soft anchors 50a, 50b, or to just to one of the two soft anchors 50a, 50b.

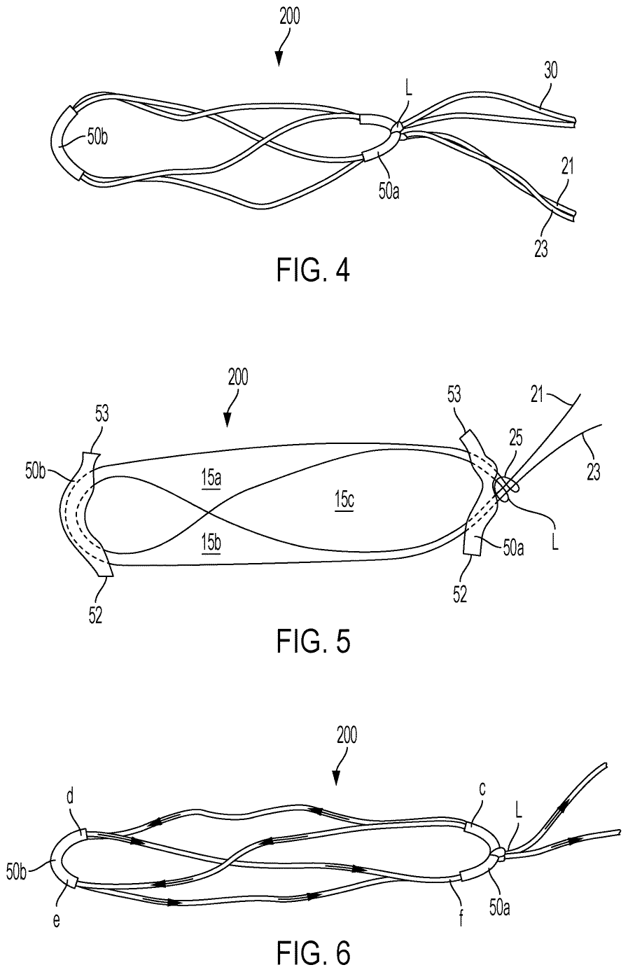

FIGS. 3 and 4 illustrate assembly 200 which is about similar to surgical assembly 100 detailed above in that it also includes two soft anchors 50a, 50b with a flexible coupler 20 passed therethrough to form flexible, closed, knotless, adjustable loops and free ends 21, 23. Surgical assembly 200 differs from surgical assembly 100 in that the flexible coupler 20 forms a suturing construct with a cross-over configuration and three adjustable, flexible, closed, knotless loops 15a, 15b, 15c. The cross-over strand configuration provides additional strength and support of the overall cerclage repair. Surgical assembly 200 also differs from assembly 100 in that it does not contain splices (can use only one end of flexible coupler 20 to form the construct) and it is a knotted construct.

In an embodiment, surgical assembly 200 can be formed by passing one of two terminal ends 21, 23 (for example, first end 21) of the flexible coupler through one of the soft-anchors 50a, 50b (for example, soft-anchor 50b, or distal anchor 50b). Terminal end 21 of flexible coupler 20 is passed through at least a portion of body 11 of fixation device 50b so that the two terminal ends 21, 23 of flexible coupler 20 reside outside the body 11 of fixation device 50b. Terminal end 21 is then passed through the other of the soft-anchors 50a, 50b (for example, soft-anchor 50a, or proximal anchor 50a). Terminal end 21 is passed a second time through body 11 of fixation device 50b and then enters the body of the fixation device 50a, at one of the two open ends 52, 53 of the fixation device 50a which is diagonally opposed to the open end exiting the fixation device 50b, through at least a portion of body 11 of fixation device 50a, and exits the fixation device 50a at about a mid-length of it, for example, at location L. Second terminal end 23 can then enter the body of the fixation device 50a, at the other of the two open ends 52, 53, through at least another portion of body 11 of fixation device 50a, and exits the fixation device 50a at about same location L.

In yet another embodiment (and as shown in FIG. 6), the cross-over configuration and the one-exit location L of the terminal ends 21, 23 can be formed by employing only one terminal end (for example, terminal end 21) and passing this end through location L on proximal anchor 50a, exiting tubular sheath a location “c” close to the one open end; passing terminal end 21 through distal anchor 50b from an open end “d” to the other open end “e”; reinserting the terminal end 21 through same location “c” in the proximal anchor 50a; passing terminal end 21 through proximal anchor 50a and exiting the anchor at location “f”; passing terminal end 21 through distal anchor 50b from open end “e” to the other open end “d”; reinserting the terminal end 21 through same location “f” within the proximal anchor 50a; and exiting terminal end 21 at location L.

The terminal ends 21, 23 are passed through proximal soft anchor 50a in different directions (for example, opposite directions) and exit the body of the soft anchor 50a at about same location L relative to the proximal soft anchor 50a. First and second terminal ends 21, 23 form first, second, and third suture loops 15a, 15b, 15c. The passes can be formed with suture passers and/or shuttle/pull devices and/or suture passing instruments such as needles, FiberLink™ loops, nitinol loops, or any suture passing device that includes an eyelet/loop for passing the flexible coupler 20. As detailed above, the terminal ends 21, 23 can be brought together into a single tail (by splicing, crimping, gluing, etc., for example) which can be subsequently cut to free up the two terminal ends.

One or more additional flexible strands 30 can be attached to construct 200 by securing the flexible strands to the soft anchors 50a, 50b or by passing the flexible strands through any of loops 15a, 15b, 15c. Additional strands can be FiberWire® suture, TigerWire® suture, FiberTape® suture tape, among many others. FIG. 4 illustrates construct 200 with only one additional flexible strand 30 attached to it. Although FIG. 4 illustrates flexible strand 30 attached to soft anchor 50a, it must be understood that the disclosure is not limited to this exemplary-only embodiment, and the disclosure also contemplates embodiments where one or more flexible strands 30 are attached to the far soft anchor 50b. In additional embodiments, one or more flexible strands 30 can be attached to each of soft anchors 50a, 50b, or to only one of the two anchors 50a, 50b.

FIGS. 5 and 6 illustrate additional views of construct 200. One or more fixation devices such as ferrule 50c of FIGS. 42 and 43 can be added in lieu of or in addition to soft anchors 50a, 50b to provide one-way locking of the construct.

Construct 200 can be knotless or knotted. If a knotted construct is desired, a sliding half-hitch knot 25 (a first knot) can be formed with the two free ends 21, 23. This sliding knot can be a pre-tied knot such as a cerclage knot or a luggage-type knot. This sliding knot (first knot) can be reinforced and/or backed up with another knot 35 to stop the sliding knot 25 from moving. The additional knot 35 (for example, another half-hitch knot or a different surgical knot such as a square knot) can be formed to lock the construct and prevent it from sliding.

Reference is now made to FIGS. 7-25 which illustrate exemplary steps of cerclage repairs 101, 102, 103 with cerclage constructs 100, 200 and exemplary hard-body fixation device 300. The repairs detailed below may be conducted with at least one all-suture implant 100, 200 and at least one hard-body fixation device 300.

Hard-body fixation device 300 may be a non-suture based fixation device such as a screw, pin, nail, hard-body anchor or fastener, or any similar device. In some implementations, the fixation device 300 may be a cannulated screw which may be fully threaded or partially threaded. In some implementations, the fixation device 300 may be a cannulated screw over a guidewire. In some implementations, the fixation device 300 may be a solid screw. In some implementations, the fixation device 300 may be a partially solid screw. As detailed below, and in one implementation, the inferior screw may be first secured to bones/grafts for stabilization of the graft, followed by the suture implant. In this manner, the disclosure allows elimination of metallic implants (such as screws and/or buttons, for example) in fixation of bone and grafts. In the case of Latarjet procedure, the superior screw tends to be the most problematic in hardware related complications. Thus, the hybrid technique of the present disclosure offers the rigidity of a screw inferiorly and the advantage of suture fixation superiorly. A hybrid construct with screw 300 and all-suture TightRope® implant system 100, 200 is unique as it offers the advantages of both systems.

The fixation device 300 may be formed of metal metals, metal alloys, or combinations of metals and non-metals. Device 300 may be formed of titanium, stainless steel or similar materials; and can be absorbable; non-absorbable; or partially absorbable. As detailed below, fixation device 300 holds the bone blocks 80, 90 together and improves the overall technique by adding strength.

The “hybrid” construct of the present disclosure uses one suture-based implant 100, 200 and one screw 300. This can be particularly helpful in situations where extra stability is desired but also where there is a need to eliminate screw complications for one of the fixation points. As noted, a particular application is the Latarjet technique or glenoid bone loss repair which require replacing the superior screw where there is a higher incidence of post-op complications using screws.

Although the embodiments below will be described with reference to one construct 100, 200 and one hard-body fixation device 300, it must be understood that the disclosure is not limited to these exemplary-only embodiments and contemplates repairs with any number of constructs 100, 200, 300. As detailed below, cerclage constructs 100, 200, 300 can be employed in both arthroscopic and open procedures.

The repairs below depict schematic representations of a first bone block or bone 80 and a second bone block or bone 90 undergoing exemplary cerclage repairs of the present disclosure. As detailed below, (i) one assembly 100, 200 is inserted and passed through two of first and second bone tunnels 81, 82, 91, 92 (through-holes 81, 82, 91, 92; passages 81, 82, 91, 92) formed within the first bone block or bone 80 and the second bone block or bone 90; and (ii) a fixation device 300 (screw 300) is inserted and passed through the other two of first and second bone tunnels 81, 82, 91, 92. In an exemplary embodiment, first bone block or bone 80 is a bone graft 80 (pre-shaped bone graft 80), and the second bone block or bone 90 is glenoid 90. In an embodiment, the first bone block or bone 80 is coracoid (pre-shaped coracoid) and the second bone block or bone 90 is the glenoid.

As detailed below, two bones 80, 90, or a bone 80, 90 and a graft 80, 90, can be joined together by drilling a single tunnel or multiple tunnels 81, 82, 91, 92. An exemplary application is for glenoid bone loss where a graft is secured to the native glenoid (and used as reference hereafter to demonstrate several methods both open, arthroscopic assisted, or arthroscopic).

Various instruments and techniques accommodate drilling single or multiple tunnels to pass one or more implants 100, 200 and assist in controlling the position/offset:
single barrel, slotted guide with side channel for pinning/rotating the guide;
double barrel, slotted guide with flange to control tunnel offset and angle (such as guide 72); and/or
double barrel, hooked arthroscopic guide.

The implant 100, 200 can be passed:
through both bones/blocks 80, 90 in sequence by either shuttling with a suture tail (i.e., arthroscopic posterior drilling and passing), or pushed with an inserter (i.e., anterior open or anterior arthroscopic); or
through a single tunnel, or through multiple tunnels.

### Pull Through Method Using Leading Suture (FIGS. 9-11)

FIG. 9: one or more bone tunnels(s) 81, 82, 91, 92 are created using a guide and drill. As noted, the tunnels can be formed through bone/graft/bone/blocks 80, 90. Lead suture 30 is shuttled through the pre-drilled bone tunnels.

FIG. 10: Implant 100, 200 is shuttled through the pre-drilled bone tunnels using the lead suture 30.

FIG. 11: The lead suture 30 is removed and the construct 100, 200 is tensioned (first by hand, then using a mechanical tensioner) to pull the bones/graft together.

FIG. 12: Screw 300 is inserted and passed through bone tunnels 81, 91 as part of final repair 101.

The steps can be repeated to insert and secure another construct 100, 200, 300 through tunnels formed within bone/graft/bone/blocks 80, 90.

The steps can be conducted by first inserting the fixation device 300 and then securing suture-based implant 100, 200. In addition, the fixation device 300 may be positioned on the bottom and the implant 100, 200 on top of the repair or vice-versa.

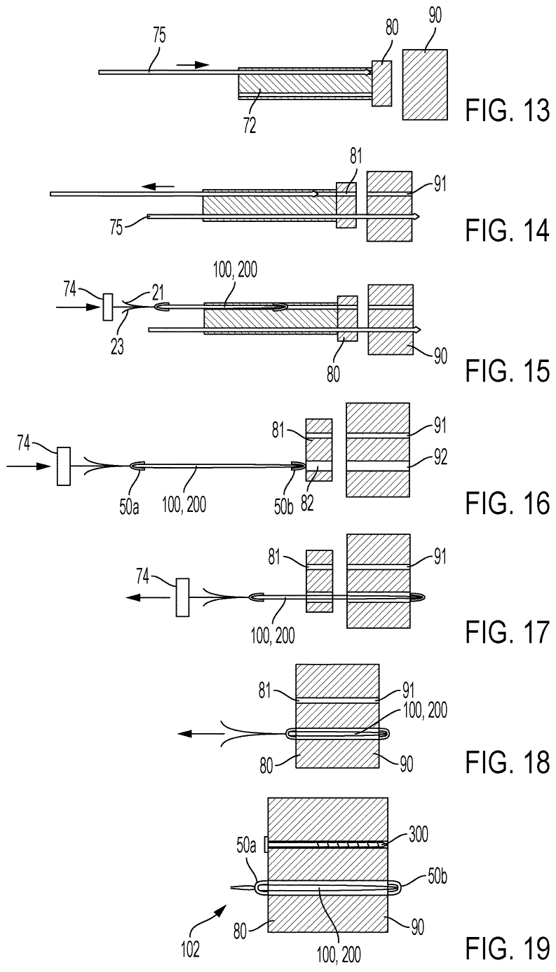

### Push Through Method Using an Inserter (FIGS. 13-19)

FIGS. 13 and 14: bone tunnels(s) 81, 82, 91, 92 are created using a guide and drills.

FIG. 15: the drills are removed and the implant 100, 200 is inserted with an inserter 74 through the guide into the drill tunnels.

FIG. 16: Optional: the implant 100, 200 is pushed by hand or malleted using an inserter 74 through the pre-drilled bone tunnels with the guide removed.

FIG. 17: whether inserted through the guide or not, the inserter is removed, leaving the implant 100, 200 in place.

FIGS. 18 and 19: The screw 300 is inserted and passed through bone tunnels 81, 91 and construct 100, 200 is tensioned (first by hand, then using a mechanical tensioner) to pull the bones/graft together as part of repair 102.

The steps can be repeated to insert and secure another construct 100, 200, 300 through tunnels formed within bone/graft/bone blocks 80, 90. The steps can be conducted by first inserting the fixation device 300 and then securing suture-based implant 100, 200. In addition, the fixation device 300 may be positioned on the bottom and the implant 100, 200 on top of the repair or vice-versa.

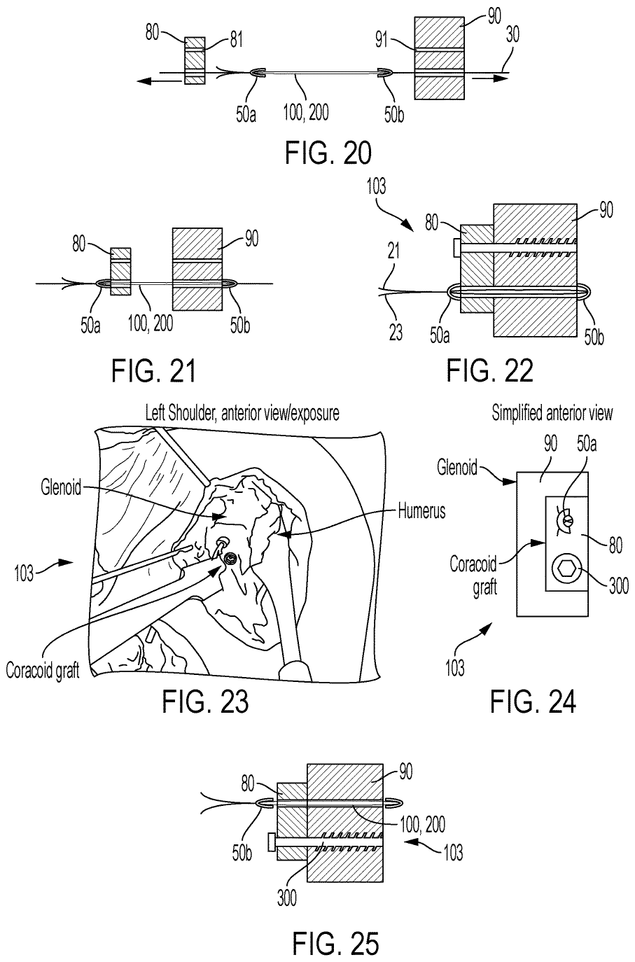

### Pull Through Method—Using Leading Sutures Both Antegrade and Retrograde (FIGS. 20-22)

The implant 100, 200 can also be passed/shuttled: antegrade through one bone (i.e. native glenoid) and retrograde through the other bone (i.e. graft); through a single tunnel, or through multiple tunnels.

FIG. 20: the implant 100, 200 is passed antegrade through one bone (e.g., native glenoid) and retrograde through the other bone (e.g., graft).

FIGS. 21 and 22: fixation device 300 is secured within bone tunnels 81, 91 and the construct 100, 200 is tensioned to pull the bones/graft together as part of repair 103. FIG. 23 illustrates a top view of repair 103 of FIG. 22 at a shoulder site.

The steps can be repeated to insert and secure another construct 100, 200, 300 through tunnels formed within bone/graft/bone/blocks 80, 90. The steps can be conducted by first inserting the fixation device 300 and then securing suture-based implant 100, 200. In addition, the fixation device 300 may be positioned on the bottom and the implant 100, 200 on top of the repair or vice-versa. If desired, multiple fixation devices 100, 200, 300 may be inserted and installed.

Knots may be formed to complete repair 101, 102, 103.

As detailed above, each assembly 100, 200, 300 can be inserted and passed through first and second bone tunnels 81, 82, 91, 92 (through-holes 81, 82, 91, 92; passages 81, 82, 91, 92) formed within a first bone block or bone 80 and a second bone block or bone 90. In an exemplary embodiment, first bone block or bone 80 is a bone graft 80 (pre-shaped bone graft 80), and the second bone block or bone 90 is glenoid 90. In an embodiment, the first bone block or bone 80 is coracoid (pre-shaped coracoid) and the second bone block or bone 90 is the glenoid.

Bone tunnels or passages 81, 82, 91, 92 can be formed, for example, by placing the bone graft 80 onto/on adjacent the glenoid 90 and drilling the first and second bone blocks or bones 80, 90 to about 2.4 mm using a posterior guide. Bone tunnels 81, 91 form a continuous first bone tunnel and bone tunnels 82, 92 form a continuous second bone tunnel, which is spaced apart from the first bone tunnel. In an implementation, fixation device 300 is passed through bone tunnels 81, 91 (formed into glenoid and bone graft). Assembly 100, 200 is passed through bone tunnels 82, 92 (formed into glenoid and bone graft). In another implementation, assembly 100, 200 is passed through bone tunnels 81, 91 (formed into glenoid and bone graft), and fixation device 300 is passed through bone tunnels 82, 92 (formed into glenoid and bone graft).

Insertion and passage of each device 100, 200, 300 through the bone tunnels 91, 81 and through bone tunnels 82, 92 can be conducted with a shuttling device or suture passing instrument such as a suture passer. Distal soft suture anchors 50b are positioned and secured on bone 90; proximal soft suture anchors 50a are positioned and secured on bone 80.

Pulling on the flexible ends 21, 23 of assembly 100, 200 allows bone graft 80 to be approximated to glenoid 90. Distal soft suture anchors 50b are bunched up and secured on the glenoid 90; proximal soft anchors 50a are bunched up and secured on a face of bone graft 80. Ends 21, 23 of flexible coupler 20 exit each of proximal soft anchors 50a. Once desired tension is achieved, additional knots such as knot 35 can be formed to stop the construct from sliding and ends 21, 23 can be cut to achieve an in-line cerclage repair. Openings 91, 92, 81, 82 house flexible, adjustable, tensionable, self-locking loops 15a, 15b, 15c.

The present disclosure provides hybrid fixation for bone to bone, with particular application for glenoid bone loss. In one embodiment, the construct includes two 2.6 mm FiberTak® sheaths used as anchors or buttons connected by a broader suture, and a threaded titanium screw 300. The pull sutures allow the surgeon to reduce the construct through a smaller 2.6 mm drill tunnel.

In another embodiment, the construct is a pre-completed cerclage construct with about 1.7 mm FiberTape® suture tape and about 2.6 mm FiberTak® sheaths. Instead of a cerclage tensioning around a bone, the tensioning occurs between the sheaths and within the bone(s)/graft, similar to tensioning between two soft buttons (with the more distal button acting in a tension-slide fashion). Both constructs can be inserted with an inserter, such as a 2.6 mm FiberTak® inserter, and a slotted guide to allow the suture to escape/exit the lumen. A ferrule can be employed in addition to or in lieu of one of the soft suture anchors 50a, 50b, to provide one way locking.

In an exemplary only embodiment, fixation devices 50a, 50b of constructs 100, 200 can be 2.6 mm FiberTak® suture anchors. Tunnels 91, 92, 81, 82 can be about 2.6 mm drill tunnels.

Constructs 100, 200, 300 can be part of various surgical kits.

In one implementation, a kit can include:
one or more implants 100, 200 provided on inserters or with shuttling/pull sutures; if two or more implants 100, 200 are part of the kit, the implants can be provided with different suture colors, and/or coated differently, for ease of suture management and to aid in suture differentiation during the surgical procedure;
one or more fixation devices 300;
two drills that can be single use (either cannulated for “pull through” technique, or solid for “push through” technique);
drill guide, which can be single-use; and
looped wires for shuttling sutures through bone tunnels.

The kit may also include guidewires and drills as detailed above.

Construct 100, 200, 300 can be employed with one or more biologics, for example, bone-to-bone biologics. Various medicinal and/or therapeutic agents, for example, antiseptics, antibiotics, drugs, pharmaceutical agents, hormones, and growth materials (for example, autogenous growth factors such as platelet-rich plasma (PRP), or autologous factors) among many others can be added at the repair site to aid in the wound closure and overall healing.

Construct 100, 200 is an adjustable, tensionable loop construct. Construct 100, 200 is an all-inside, self-locking, adjustable construct. Construct 100, 200 can be a suture/soft anchors construct in the form of an orthopedic implant construct which can be utilized to attach or re-attach a first tissue to a second tissue, for example, bone to bone, graft to bone, and/or bone to tissue, among others. The construct includes a single strand of suture run through two soft suture anchors, with ends of the suture exiting a single soft suture anchor to create a tensionable, self-locking, adjustable loop, anchoring construct. The construct can be provided assembled, disassembled, as part of a kit with fixation device 300, and with or without additional instruments (such as suture passers, inserters, guides), fixation devices and/or flexible couplers, to facilitate passing through tunnels and/or bone sockets and/or openings.

The hybrid arthroscopic tissue repair devices/constructs of the present disclosure can be employed for any tissue positioning and/or tissue adjustment applications, for example, in fixation of bone to bone (such as small joint applications, or acromioclavicular joint fixation techniques) which employ two fixation devices (two flexible suture anchors or two soft buttons) joined by a plurality of continuous suture loops formed by a continuous flexible coupler, and at least one fixation device 300. In exemplary embodiments only, construct 100, 200, 300 of the present disclosure can be employed in a method of bunion repair and/or in a method of Lisfranc repair. Similarly, construct 100, 200, 300 can be employed in any method of fixation of bone.

The hybrid screw/suture implant with the self-locking mechanism can be utilized in surgical procedures such as rotator cuff repair, Achilles tendon repair, patellar tendon repair, ACL/PCL reconstruction, hip and shoulder reconstruction procedures, AC joint reconstruction, syndesmosis reconstruction, quad/patellar tendon rupture repair, hallux-valgus repair, proximal and/or distal biceps tendon repair, humerus and radius repair, and any other tendon repair to bone, among many others, all conducted in a knotless manner.

A surgical assembly 100, 200 includes a plurality of fixation devices 50a, 50b, 50c and a knotless, adjustable, self-locking tensionable construct 20 loaded on the fixation devices 50a, 50b. The tensionable construct 20 includes at least one flexible coupler 20 having a first end 21 and a second end 23; and at least first and second closed, adjustable, continuous, flexible loops 15a, 15b, 15c. Fixation devices 50a, 50b are all-suture soft anchors. Surgical assembly 100, 200 can connect first tissue 80 to second tissue 90. First tissue 80 can be a bone, bone fragment, osteochondral fragment, or bone graft; second tissue 90 can be bone. Distal fixation device 50b can reside and be secured within opening 91, 92 formed within second tissue 90. Proximal fixation device 50a can reside and be secured within openings 81, 82 of first tissue 80. Terminal ends 21, 23 are pulled to decrease the distance between the two fixation devices 50a, 50b and decrease the length and perimeter of the flexible, adjustable, closed, knotless loops 15a, 15b. Surgical assembly 100, 200 can consist essentially of suture. Surgical assembly 100, 200 can consist essentially of elastic suture. Surgical assembly 100, 200 may be employed with one or more hard-body fixation device 300.

A method of tissue repair 101, 102, 103 comprises inter alia the steps of: (i) passing a flexible coupler 20 through sheaths of two fixation devices 50a, 50b, to form a tensionable, self-locking, adjustable construct 100, 200 with at least two loops 15a, 15b, 15c and with ends 21, 23 of the flexible coupler exiting a fixation device 50a; (ii) securing one of the two fixation devices 50a, 50b in a first tissue 90; and (iii) securing the other of the two fixation devices 50a, 50b in a second tissue 80. The method may include the step of securing a hard-body fixation device 300 within the first and second tissues 90, 80. The method can further include tensioning the construct by pulling on terminal ends 21, 23 of flexible coupler 20. The repair can be a cerclage repair. The step of securing the hard-body fixation device 300 may be conducted before or after steps (i)-(iii).

A method of cerclage repair 101, 102, 103 comprises inter alia the steps of (i) passing a cerclage construct 100, 200 consisting essentially of two or more flexible loops 15a, 15b, 15c attached to soft anchors 50a, 50b through first and second bone segments 80, 90 (bones, bone blocks, bone grafts, bone fragments); and (ii) securing a fixation device 300 within the first and second bone segments. Step (ii) may be conducted before step (i). The method may include repeating the steps (i) and (ii) for additional cerclage constructs 100, 200, 300.

The cerclage constructs 100, 200, 300 and methods of the present disclosure provide apparatus and methods for tissue repair, for example, bone to bone repair, or other tissue (such as graft) to bone repair. The surgical cerclage constructs allow for simplified cerclage repairs and help simplify shuttling and managing of cerclage suture. The surgical constructs 100, 200 are similar to Fiber TightRope® all-suture suspensory fixation implants. The constructs 100, 200 minimize the use of metallic components (screws, buttons, etc.) where bone to bone fixation is required, i.e., bone graft fixation to the glenoid for glenoid bone loss cases (Latarjet procedure, Bristow procedure, free bone block, etc.). Suture tape can run between two soft suture button implants or locking ferrule top hap and a single soft button that is configured to tension on one end and slide on the opposite end. The constructs 100, 200 can be 100% suture-based and do not rely on traditional finger-trap fixation. Constructs 100, 200 have a unique suture assembly that allows for smooth and even suture sliding at the “far” button. Constructs 100, 200 may be employed with one or more hard-body fixation devices 300.

Flexible coupler 20 can be formed of various flexible materials and strands such as round suture, flat suture, ribbon, or flat tape (for example, suture tape) or combination of suture and tape. Exemplary materials can include suture, silk, cotton, nylon, polypropylene, polyethylene, ultrahigh molecular weight polyethylene (UHMWPE), polyethylene terephthalate (PET), and polyesters and copolymers thereof, or combinations thereof. Flexible strand/coupler 20 can have cross-sections of various forms and geometries, including round, oval, rectangular, or flat, among others, or combination of such forms and geometries. In an exemplary embodiment only, flexible coupler 20 can be formed of a high strength suture material such as FiberWire® suture, sold by Arthrex, Inc. of Naples, Fla., and described in U.S. Pat. No. 6,716,234, the disclosure of which is incorporated by reference herein. FiberWire® suture is formed of an advanced, high-strength fiber material, namely ultrahigh molecular weight polyethylene (UHMWPE), sold under the tradenames Spectra® (Honeywell International Inc., Colonial Heights, Va.) and Dyneema® (DSM N.V., Heerlen, the Netherlands), braided with at least one other fiber, natural or synthetic, to form lengths of suture material. Flexible coupler 20 can be braided or multi-filament suture such as FiberTape® suture tape (as disclosed in U.S. Pat. No. 7,892,256, the disclosure of which is incorporated in its entirety herewith). If suture tape is employed, the tape can have sections with different tapers.

Flexible coupler 20 can be also formed of a stiff material, or combination of stiff and flexible materials, particularly for the regions of the couplers that are passed/spliced through the body of the coupler and depending on whether they are employed with additional fixation devices. Various regions, or sections of flexible coupler 20 can be coated and/or provided in different colors for easy manipulation during the surgical procedure. Flexible coupler 20 can be formed partially or totally of collagen-coated suture. Flexible coupler 20 can be formed of radiopaque suture. Flexible coupler 20 can be provided with tinted tracing strands, or otherwise contrast visually with the sheath 51 of the soft suture anchors 50a, 50b, which remains a plain, solid color, or displays a different tracing pattern, for example. Easy identification of suture in situ is advantageous in surgical procedures, particularly during arthroscopic surgeries, endoscopic and laparoscopic procedures.

Various elements of construct 100, 200 can be also coated (partially or totally) with wax (beeswax, petroleum wax, polyethylene wax, or others), silicone (Dow Corning silicone fluid 202A or others), silicone rubbers (Nusil Med 2245, Nusil Med 2174 with a bonding catalyst, or others) PTFE (Teflon, Hostaflon, or others), PBA (polybutylate acid), ethyl cellulose (Filodel) or other coatings, to improve lubricity of the suture or tape, knot security, pliability, handleability or abrasion resistance, for example. If desired, at least a region of the flexible strands/coupler 20 can be coated, impregnated, or otherwise stiffened with a material such as plastic, for example.

The term “high strength suture” is defined as any elongated flexible member, the choice of material and size being dependent upon the particular application. For the purposes of illustration and without limitation, the term “suture” as used herein may be a cable, filament, thread, wire, fabric, or any other flexible member suitable for tissue fixation in the body.

What is claimed is:

1. A cerclage construct comprising:
a flexible coupler connected to a first soft anchor and to a second soft anchor by a plurality of closed, continuous, flexible, knotless, adjustable loops with an adjustable perimeter; and
a hard-body fixation device.

2. The cerclage construct of claim 1, wherein the flexible coupler passes at least once through a first tubular sheath of the first soft anchor and at least once through a second tubular sheath of the second soft anchor to form the plurality of closed, continuous, flexible, knotless, adjustable loops with an adjustable perimeter.

3. The cerclage construct of claim 2, wherein the flexible coupler has ends that exit one of the first tubular sheath and the second tubular sheath at a same location.

4. The cerclage construct of claim 3, wherein the same location is located at about a mid-length of one of the first tubular sheath and the second tubular sheath.

5. The cerclage construct of claim 1, wherein each of the first soft anchor and the second soft anchor is an all-suture anchor, and the flexible coupler is suture tape.

6. The cerclage construct of claim 1, wherein the first soft anchor is configured to be secured on a surface of a first bone, and wherein the second soft anchor is configured to be secured on a surface of a second bone.

7. The cerclage construct of claim 1, wherein the hard-body fixation device is a screw, a pin, a nail, or an anchor.

8. The cerclage construct of claim 1, wherein the hard-body fixation device is formed of metal or metal alloys.

9. The cerclage construct of claim 1, wherein the construct is part of a kit.

10. A surgical assembly for bone cerclage repairs, comprising:
a first fixation device and a second fixation device, each consisting essentially of suture; and a flexible coupler passed through the first fixation device and through the second fixation device, the flexible coupler forming in a knotless manner a plurality of continuous, adjustable, tensionable, flexible loops extending between the first fixation device and the second fixation device; and
a screw.

11. The surgical assembly of claim 10, wherein the screw is threaded.

12. The surgical assembly of claim 10, wherein the screw is formed of titanium or titanium alloys.

13. The surgical assembly of claim 10, wherein each of the first fixation device and the second fixation device includes a tubular sheath with two open ends.

14. The surgical assembly of claim 10, wherein the flexible coupler forms two continuous, adjustable, tensionable, flexible loops extending between the first fixation device and the second fixation device.

15. The surgical assembly of claim 10, wherein the bone cerclage repair is glenoid bone loss repair.

16. A method of tissue repair comprising:
inserting a hard-body fixation device within a first tissue and a second tissue; and
inserting a first soft anchor through the first tissue and inserting a second soft anchor through the second tissue, wherein the first soft anchor is knotlessly connected to the second soft anchor by a flexible coupler forming a plurality of continuous, closed, adjustable, tensionable, flexible loops extending between the first soft anchor and the second soft anchor.

17. The method of claim 16, further comprising:
securing the first soft anchor in the first tissue; and
securing the second soft anchor in the second tissue.

18. The method of claim 17, further comprising:
passing at least once the flexible coupler through a first tubular sheath of the first soft anchor;
subsequently, passing at least once the flexible coupler through a second tubular sheath of the second soft anchor to form the plurality of continuous, closed, adjustable, tensionable, flexible loops;
subsequently, passing each of two ends of the flexible coupler through one of the first soft anchor and the second soft anchor;
forming a half-hitch knot with the two ends;
securing the first soft anchor to an inserter;
passing the first soft anchor through the first tissue;
securing the first soft anchor into a first opening in the first tissue at a first location;
securing the second soft anchor to an inserter;
passing the second soft anchor through the second tissue; and
securing the second soft anchor into a second opening in the second tissue at a second location.

19. The method of claim 16, wherein the first tissue is bone or bone graft and the second tissue is bone.

20. The method of claim 16, wherein the tissue repair is glenoid bone loss repair.

21. The method of claim 16, wherein the tissue repair is rotator cuff repair, AC joint repair, syndesmosis repair, Achilles tendon repair, patellar tendon repair, ACL/PCL reconstruction, hip and shoulder reconstruction, AC joint reconstruction, syndesmosis reconstruction, quad/patellar tendon rupture repair, or hallux-valgus repair.
